(19) United States
(12) Patent Application Publication
(10) Pub. No.: US 2025/0356078 A1
(43) Pub. Date: Nov. 20, 2025

(54) DIGITAL MENTAL TWIN

(71) Applicant: ZENTRALINSTITUT FÜR SEELISCHE GESUNDHEIT, Mannheim (DE)

(72) Inventor: Andreas MEYER-LINDENBERG, Mannheim (DE)

(73) Assignee: ZENTRALINSTITUT FÜR SEELISCHE GESUNDHEIT, Mannheim (DE)

(21) Appl. No.: 18/872,595

(22) PCT Filed: Jun. 8, 2023
(86) PCT No.: PCT/EP2023/065424

§ 371 Date: Dec. 6, 2024

Related U.S. Application Data
(60) Provisional application No. 63/350,115, filed on Jun. 8, 2022.

(51) Int. Cl. G06F 30/20

(52) U.S. Cl. CPC G06F 30/20

(57) ABSTRACT

This document teaches a computer-implemented system for simulating a mental state of a mind of a human. The system comprises a memory including a plurality of instructions, virtual data storage and human mental data storage. The mental data storage stores mental data relating to the mental state of the mind of the human. At least one processor can execute ones of the plurality of instructions to generate at least a digital twin of the mind of the human. The digital twin includes one or more models of the mind of the human based on human mental data. The one or more models of the mind are stored in a virtual data storage and enable analysis of the mind of the human by the at least one processor within the virtual data storage. The digital twin includes a link between the mental data storage and the virtual data storage.

### CROSS-REFERENCE TO RELATED APPLICATIONS

This application claims the benefit of the filing date of U.S. provisional application No. 63/350,115, the disclosure of which is hereby incorporated herein by reference in its entirety.

### BACKGROUND OF THE INVENTION

### Field of the Invention

The field of the invention relates to a method and a computer-implemented system for creating, maintaining, updating, generating predictions from, and otherwise utilizing a regularly updated representation of the state of mind of a human.

### Brief Description of the Related Art

A digital twin is a virtual representation of an object or process that serves as a real-time digital counterpart reflecting the state of the object or process through being updated by information from the object's environment and the object itself. The concept of the ‘digital twin’ originated in engineering science and provides a framework to employ data-driven health care practices, as well as their conceptual and ethical implications for therapy and preventative action. When applied to human beings, current concepts of digital twins build an in-silico representation of an individual that dynamically reflects the body of the human being and its physiological status. The concept of a digital twin has been emerging slowly in the health care industry (as outlined by Bruynseels K, Santoni de Sio F, van den Hoven J. Digital twins in Health Care: Ethical Implications of an Emerging Engineering Paradigm. Front Genet. 2018 Feb. 13; 9:31. doi: 10.3389/fgene.2018.00031. PMID: 29487613; PMCID: PMC5816748).

US Patent Application No. 2020/203020 teaches a method of developing a personalized digital model (or digital twin) of at least part of the anatomy of a person with a computer system comprising a processor arrangement and a communication module under control of the processor arrangement. The method comprises, with said processor arrangement, receiving input data relevant to an actual physical condition of at least part of the anatomy of the person with the communication module; searching a database of digital models modelling different physical conditions of said at least part of the anatomy and selecting a digital model from said database that most closely matches the actual physical condition of the at least part of the anatomy of the person based on at least some of the received input data. The selected digital model is processed by developing the modelled physical condition of the selected digital model with a physiological development model associated with said selected digital model based on the received input data such that the developed modelled physical condition more closely resembles said actual physical condition in accordance with said input data.

A computerized method for healthcare data management using a digital twin of the individual patient based on the health information related to the individual patient is known from US Patent Application No US 2020/303047. This patent publication teaches a digital twin of an individual patient being a digital representation of at least one health state of the individual patient and forming, using the healthcare data system computing device, a digital twin of the population of patients based on the health information related to the population of patients. The digital twin described in this application can aid in determining whether the usage profile of a pharmacological agent by the human patient is indicative of potential misuse of the controlled medication. Furthermore, in response to determining potential misuse of the controlled medication, the method includes transmitting a notification that indicates the potential misuse by the patient. However, this application does not consider the mental state of the patient.

A system for management of a stress level and mental health of a human body is known from the PCT application No. WO 2019/012471. The system of this application has one or more body sensors and a primary processing unit that runs an artificial intelligence system. The body sensors are adapted to measure at least one of a physiological parameter of the human body, body movement of the human body, or heat expenditure of the human body or combination thereof, and to generate a body data periodically or in real-time. The primary processing unit is adapted to receive and process the body data and adapted to determine at least one of the health and the stress level of the human body. The primary processing unit is adapted to provide therapies and give insights about the effectiveness of psychological therapies including CBD, meditation, and mindfulness in a quantitative manner.

The prior art discloses digital twins or simulations to describe physical parameters of a human body. It would be useful to be able to simulate and continuously represent the human mind to assess and predict the mental state of a human.

### BRIEF SUMMARY OF THE INVENTION

The present invention is a computer-implemented system for simulating the state of a mind of a human. The system comprises a memory including a plurality of instructions, virtual data storage and mental data storage. The mental data storage stores user data relating to the state of the mind of the human who is being represented by the system. At least one processor can execute ones of the plurality of instructions to generate at least a digital twin of the mind of the human. The digital twin includes one or more models of the mind of the human, based on mental data. The one or more models of the mind are stored and updated in a virtual data storage and enable analysis of the mind of the human by the at least one processor within the virtual data storage. The digital twin includes a link between the mental data storage and the virtual data storage

In a further aspect, the system comprises interfaces to make a representation of the state of mind available to users outside the system. This may include a graphical user interface to provide visualization of and access to the human mental data based on an output from the digital twin. This may also include an application interface to make such data available to other software or platform uses. The system may further comprise one or more notification devices for triggering a notification form the digital twin and a prediction device for predicting the mental state of the human.

A computer-implemented method for simulating a mental state of a mind of a human is also disclosed. The method comprises applying one or more simulation parameters to a digital twin, wherein the digital twin comprises one or more models of the mind of the mind of the human. The digital twin enables simulating the mind of the human. The one or more simulation parameters cause the digital twin to generate a digital twin output that simulates behavior of the mind of the human, e.g., one or more subsequent states of the mind. The applying of the one or more simulation parameters to the digital twin may comprise inputting the one or more simulation parameters into the one or more models of the mind. The simulating of the mental state of the mind of the human may comprise calculating a trajectory of the mental state of the mind of the human in a state space (or phase space). The trajectory of the mental state of the mind of the human in the state space (or phase space) may depend on the simulation parameters inputted into the digital twin of the mind of human. The system can receive and is updated by information from sensors, the user or external data sources that modify the state of mind, such as the presence of other people, location, or heart rate.

The digital twin output comprises, for example, a graphical depiction of mental state, a mental health prediction or a connection to an avatar generator.

In a further aspect, the method enables comparing the digital twin output with a response of another human or to an ensemble of digital mind twins.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE FIGURES

For a more complete understanding of the present invention and the advantages thereof, reference is now made to the following description and the accompanying drawings, in which:

FIG. 1 shows an overview of a digital twin.

FIG. 2 shows an overview of a method.

FIG. 3 shows a concept for a baseline questionnaire used at the beginning of a study.

FIG. 4 shows demographic characteristics, psychological characteristics, Ecological Momentary Assessment and neuroimaging data collected during the study referred to in FIG. 3.

### DETAILED DESCRIPTION OF THE INVENTION

The invention will now be described on the basis of the drawings. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

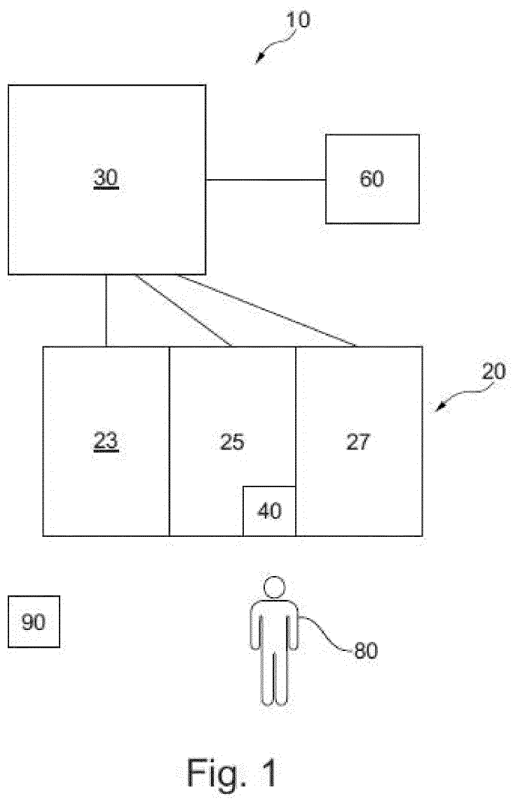

FIG. 1 shows an overview of the system 10 for simulating a mental state of a mind of a human. The system 10 comprises a memory 20 which includes storage location for storing a plurality of instructions 23, virtual data storage 25 and mental data storage 27. The system 10 has at least one processor 30 which can execute ones of the plurality of instructions 23 stored in the storage location to generate a digital twin 40 of the mind of the human, as will be explained later. The virtual data storage 25 is used by the processor 30 to produce the digital twin 40. The mental data storage 27 stores mental data relating to the mental state of the human.

In one aspect of the disclosure, the mental data storage 27 may store populational mental data about the mental states of other humans, including mental data of healthy humans and/or humans suffering from mental diseases and/or physical diseases. In another aspect, the mental data relating to, e.g., representing, the mental state of the human are adaptable and can be defined and/or amended, for instance dependent on a monitoring of an onset or a development of a mental disease or mental disorder. The defining and/or amending of the mental data comprises updating the mental data. The updating of the mental data may comprise updating the mental data on a daily basis or an hourly basis, e.g., dependent on the mental state of the human being simulated and on an availability of newly collected mental data. The updating may occur at predefined times or be prompted by the human or an event. The defining and/or amending of the mental data may depend on the human and/or the mental state of the human. In a further aspect, the mental data may relate to one or more of mood, affect, arousal, alertness, energy level of the human, level of hunger, thirst, aggression, or fear of the human, but are not limited thereto.

In yet a further aspect, the mental data storage 27 may further include one or more of physiological data, environmental (or contextual) data, and/or personal data. The physiological data may include data relating to sleep quality, physical activity, bodily exertion, electrodermal activity, electrocardiogramd/or neuroimaging. The environmental (or contextual) data may include location data (e.g., GPS data), data relating to social interactions such as emails, text message, social media posts, voice mails, calls, weather conditions, voice patterns, motion patterns. The personal data may include sleep duration, amount of steps walked, time spent outdoors, time spent in green spaces, time spent exercising, medical data (e.g., from an electronic health record) such as data relating to a behavior therapy or an administration of a medication. The mental state of the human may be directly or indirectly linked to the physiological data, the environmental (or contextual) data, and/or the personal data.

In one aspect, the mental data, the physiological data, the environmental (or contextual) data, and/or the personal data may in part be measured or inferred from objective data that collected by sensors 80, 90 (for example, energy level from movement patterns) or by user response. The data may be collected through the method of ecological momentary assessment (EMA) (see Reichert M, Gan G, Renz M et al. (2021) Ambulatory assessment for precision psychiatry: Foundations, current developments and future avenues. Experimental neurology: 113807). The EMA may comprise using motion sensors 90, location data (e.g., GPS), data relating to a usage of a digital app (e.g., based on number of logins or parts of the digital app used).

The digital twin 40 includes one or more models 50 of the mind of the human and

is created by the processor 30 in the virtual data storage 25. The digital twin 40 also includes a link between the mental data storage 27 and the virtual data storage 25 which enables the digital twin 40 to create in the virtual data storage 25 a simulation of the mind of the human.

The system 10 also has a graphical user interface 60 displayed, for example, on a computer screen, a tablet, or a smartphone. The graphical user interface 60 provides a visualization of the simulated mental state, which is based on the data stored in the mental data storage 27 and is output from the digital twin 40. The graphical user interface 60 also enables a user of the system, such as a medical practitioner, to interact with the digital twin 40. For example, the user may wish to enter data into the system 10 to understand how this might affect the mental state of the human being. The digital twin 40 can simulate the response of the patient.

The system 10 could also be connected to external applications or platforms. For example, a graphics engine 62 for creating avatars in the metaverse. An avatar includes one or more graphical representations of a person and can be in the form of a person or a creature. The graphics engine 62 includes a generator for creating the avatar of the human and can be fed with parameters from the digital twin 40 to enable a better reaction of the avatar in the metaverse. For example, the parameters could influence the facial expression or body language of the avatar. As another example, information about mental state could be made available externally to suggest a mood-appropriate service or product or a call to a therapist.

The physiological data may be collected from a at least one physiological sensor 80 attached to a living human being. The physiological data may be fed into the system 10 and stored in the mental data storage 27. The physiological data may be applied to and used by the digital twin 40 to simulate the response of the human. The at least one physiological sensor 80 measures, for example, heart rate, blood pressure, heart rate variability, skin conductions, pupillary reactivity, voice activity, motor movement, eye movement etc.

At least one environmental sensor 90 can also be connected to the system 10. The at least one environmental 90 measures, for example, light intensity, temperature, air pressure, humidity, the proximity of other human beings or devices worn by the other humans.

In one aspect, a mobile device, such as a smartphone, may comprise the at least one physiological sensor 80 and/or the at least one environmental sensor 90. The mobile device may be used to generate some of the physiological data from the at least one physiological sensor 80, some of the environmental (or contextual) data from the at least one environmental sensor 90, and/or the personal data. The mobile device can record using GPS the distance walked by the human carrying the device. The mobile device can further detect interactions with other human beings by interacting with further mobile devices carried by the other human beings. These interactions are recorded with a time stamp indicating the length of time of the interaction and the time of the interaction. The mobile device can also ask the human questions to determine the human's well-being and record answers to the questions. The questions could also be asked by another human. The physiological data, the environmental data, and/or the personal data, collected by the mobile device, will be stored in the mental data storage 27. In another aspect of the disclosure, the mobile device comprises a wearable, such as a smart watch, a smart band, a smart ring, a smart chest strap, and/or XR goggles.

Other personal and/or environmental data that can be recorded by the mobile device include, but are not limited to, ambulatory data, such as speed of walking or acceleration, as well as geolocation.

The digital twin 40 is stored in the virtual data storage 25 as well as on the device hosting the local processor (such as the user's smartphone). The representation is done by a recurrent neural network (RNN), e.g., a deep recurrent neural network (deep RNN) and/or a piecewise-linear recurrent neural network (PLRNN), that is pretrained by a range of datasets characterizing the human's mental state as a function of the human's behavior. Training is done using code, implemented in MATLAB or another coding language. For example, datasets have been built combining regularly assessed user's wellbeing with, e.g., continuous or regular, measurements of movement to demonstrate positive impacts of activity on mood, or datasets combining satellite-imaging-based information about green space with GPS-based geolocation to show improved mood when users encounter green space in urban contexts.

The inputs of this deep neural network are the user inputs, the sensor data, and external datasets linked by those informations (for example, information about the kind of local environment drawn from geographic information based on sensor-delivered current location of the user.

The outputs of the trained neural network are a set of mental parameters. Training of the neural network is performed in the virtual data storage 25. The trained neural network thus trained provides an updated representation of the mental state and forms the digital twin 40. This representation is updated whenever user input, sensor and environmental data become available. The network training uses novel trainings that optimize the network-based reconstruction and prediction of the human mind as a dynamical system, as published in previous work from the inventor. This means that after training the network reproduces the underlying dynamics of the mental states and in this sense becomes a functional mirror that can be utilized for prediction, as described below.

A trained RNN model captures a discrete time-dynamical system Xₜ=F_{θ}(xₜ₋₁, uₜ), where xₜ represents a state of the dynamical system, e.g., the brain of the human, that evolves in time according to the recursive map F_{θ}, parameterized by a vector of parameters θ, and uₜ denotes a time-series of external inputs (regressors) into the system. Again, while in traditional statistics F_{θ} is usually linear, giving rise to the important class of autoregressive moving-average (ARMA) models, for RNNs Fe is (highly) nonlinear. If the temporal sequences on which the RNN is to be trained have a known and fixed maximum length T, Fe may be “unwrapped” in time and a technique called “Back-Propagation through time (BPTT)” may be employed for training [20, 182]. An important property of RNNs is that, beyond their application as time series analysis and prediction tools, they can generate sequences and temporal behavior themselves autonomously, thus making them powerful AI devices also in situations that require goal-directed behavior and planning. The dynamical system enables relating an activity evolution in the RNN to brain processes based on attractor states, oscillations (limit cycles), or chaotic dynamics. The dynamical system undergoing a bifurcation may be associated with a transition to a desired one or an undesired one of the mental state of the human. In the case of the transition to an undesired one of the mental state, the system 10 and/or the digital twin 40 may trigger a warning. The warning may for example be displayed by the graphical user interface 60. The warning may also be provided to the application interface, e.g., for transmission to the therapist, e.g., in the form of notifications 64 (see below).

The RNN may be inferred from the mental data, the physiological data, the environmental (or contextual data), and/or the personal data. In particular, the method may comprise collecting time series of the mental data, the physiological data, the environmental (or contextual data), and/or the personal data, which may be referred to as a multimodal empirically observed time series. The RNN may be inferred from the multimodal empirically observed time series using maximum-likelihood or Bayesian inference. The inference may be based on a model p(yₜ|θ), in which yₜ are the measured time-series of the mental data, the physiological data, the environmental (or contextual data), and/or the personal data, and θ is the vector of parameters defining the recursive map F_{θ} representing the evolution of the dynamical system represented by xₜ. The inference may employ evidence-lower bound (ELBO) in order to approximate a calculation of an integral.

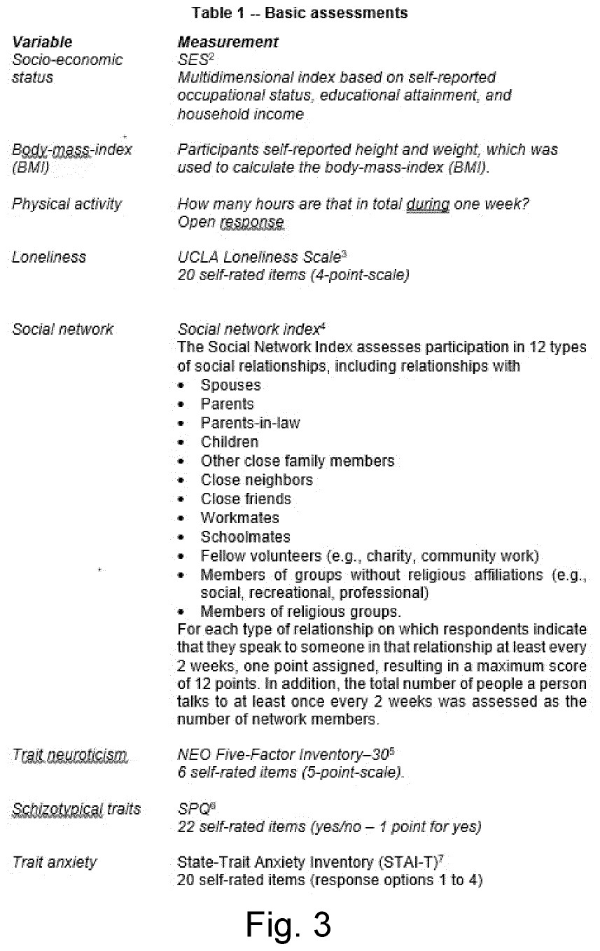

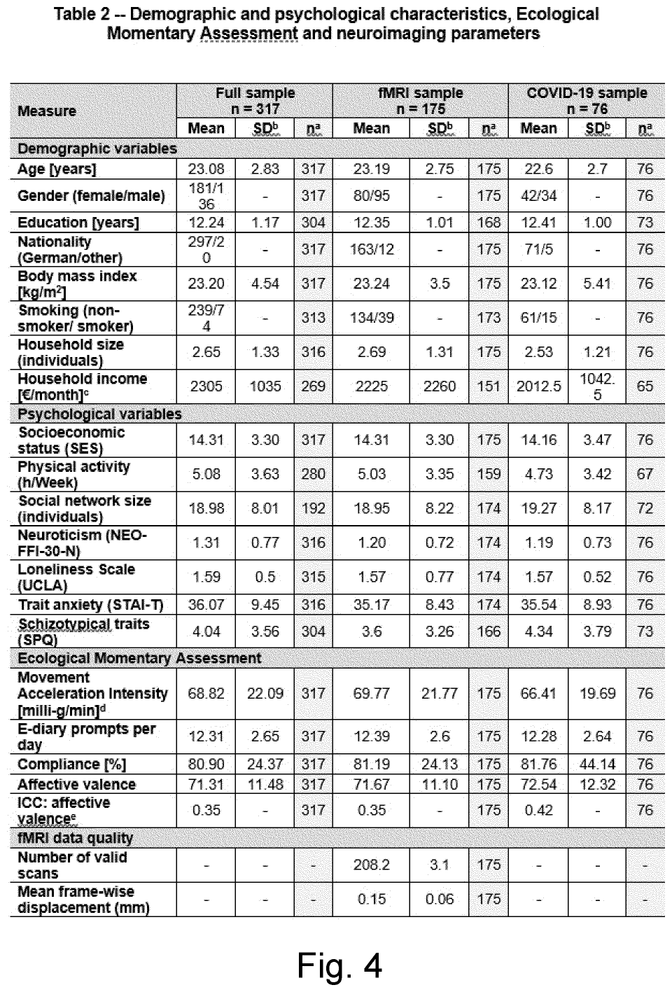

One example of the multimodal empirically observed time series is now described used for training the RNN is now described. 356 healthy participants between 18 and 28 years were recruited. All participants wore an accelerometer and a study smartphone and completed additional baseline questionnaires (see Table 1 in FIG. 3), which the participants completed including sociodemographic information, height and weight, and several psychological assessments, as detailed in FIG. 3. Following established procedures, we excluded participants if the following criteria applied: (i) severe technical problems with the accelerometer such as a prematurely terminated measurement (N=28), (ii) e-diary compliance below 30% (N=2), or (iii) missing questionnaire data (N=9). The final sample consisted of 317 healthy participants (57.09% females) with a mean age of 23.08 years (SD=2.83; see Table 2 in FIG. 4).

Table 2 presents results for three samples named “Full sample”, “fMRI sample”, and “COVID-19 sample”. In Table 2 shown in FIG. 4, the columns with the sample size n indicate the number of individuals for which the information for the corresponding variable was available. SD means standard deviation. The household income was assessed as monthly household income after taxes in 13 ordinal categories, i.e., 1) less than 500 €, 2) 500-749 €, 3) 750-999 €, 4) 1000-1249 €, 5) 1250-1499 €, 6) 1500-1749 €, 7) 1750-1999 €, 8) 2000-2249 €, 9) 2250-2499 €, 10) 2500-2999€, 11) 3000-3999€, 12) 4000-4999€, and 13) more than 5000€. For the descriptive comparison of the samples in this table we assigned category means to individuals, e.g., a value of 624.5 € to a participant belonging to the second category. Values of Movement Acceleration Intensity were averaged across participants and the study week, respectively. For affective valence, intra class correlation coefficients (ICC) were used to calculate variance estimates of our outcome variables: In the study 35.0% of the variance in affective valence can be attributed to within-subject variation.

Participants were informed about the study, provided written consent, and received monetary compensation for participation at the end of the study. Participants received an extensive in-person technical briefing, including testing, and thereafter carried a study smartphone and an accelerometer for seven consecutive days in their everyday life. After one week, participants returned the devices and reported on their most important locations visited. To enhance participant's recall, we applied an established procedure similar to the Day Reconstruction Method 1. Briefly, we used a time-stamped digital map (movisens Geocoder) that showed all geolocations visited and routes covered (tracked via smartphones). Participants were asked to label all situational contexts retrospectively (such as being at home, at work, out with friends). These location labels were later assigned to three categories: ‘home’, ‘work’ and ‘other’, representing the situational context. Prior to these procedures, participants completed a questionnaire battery including sociodemographic information, height and weight, and several psychological assessments, as detailed in FIG. 3.

For assessing physical activity, participants wore a triaxial accelerometers (Move II or Move III; movisens GmbH, Germany) for seven consecutive days during waking hours on the right hip. The accelerometer captures movements of as much as +8 g with a resolution of 12 bits and a sampling frequency of 64 Hz and appropriately assesses human physical activity. To compute Movement Acceleration Intensity, i.e., the vector magnitude of the acceleration in milli-g [(g)/1000] assessed at the three sensor axes, we used the software DataAnalyzer by movisens GmbH (version 1.6.12129). In short, gravitational components were eliminated by a high-pass filter (0.25 Hz), and artifacts (e.g., vibrations when cycling on a rough road surface or shocks of the sensor) were eliminated by a low-pass filter (11 Hz). To differentiate light physical activity, we computed the metabolic equivalent of task (MET), a measure of energy expenditure and defined as the ratio of work metabolic rate to a standard resting metabolic rate of 1.0 (4.184 kJ)*kg-1*h-1, with 1 MET representing the resting metabolic rate obtained during quiet sitting. Based on METs, activities can be categorized, e.g., into light-intensity physical activity (1.6-2.9 METs). We calculated the METs using the software DataAnalyzer (movisens GmbH, Germany). Prior to the MET calculations, gravitational components were eliminated by a high-pass filter (0.25 Hz), and artifacts (e.g., vibrations when cycling on a rough road surface or shocks of the sensor) were eliminated by a low-pass filter (11 Hz). The METs were calculated in two steps: First, an activity class was estimated based on acceleration and barometric signals. Based on the detected class, the corresponding model for MET calculations was chosen, and based on movement acceleration, altitude change extracted from barometric data, age, gender, weight, and height, MET values were calculated, established procedures described elsewhere.

E-diaries and the sampling strategy were implemented via the ecological momentary assessment software movisensXS, version 0.6.3658 (movisensXS12). After thorough instruction, participants carried a smartphone (Motorola Moto G, Motorola Mobility) for seven consecutive days and were prompted via an acoustic, visual, and vibration signal to fill in the e-diary multiple times per day. The prompt could be postponed for 5, 10, or 15 minutes. The prompts were triggered based on a mixed time- and location sampling scheme that is superior to traditional time-based sampling schemes (e.g., missed rare events) and increases the within-person variance of interest. On each day during the study week, e-diary prompts were triggered between 7.30 AM and 10.30 PM with a minimum time-interval of 40 minutes and a maximum of 100 minutes between two e-diary prompts. This resulted in a total of 9 to 23 e-diary prompts per day. The location-based trigger algorithm monitored the distance between the participants' current and previous locations continuously. When a distance larger than 500 meters was covered, a prompt was triggered. In addition, participants were triggered at two fixed times every day (8 AM and 10.20 PM).

To assess affective valence, both studies used an established two-item short scale with appropriate reliability and sensitivity to measure within-subject fluctuations of mood. The two items were presented as bipolar scales with a score range from 0 to 100 (′content′ to ‘discontent’; in German, ‘zufrieden’ to ‘unzufrieden’ and ‘unwell’ to ‘well’; in German, ‘unwohl’ to ‘wohl’) in reversed polarity at the edges of two computerized visual analogue scales. The two item scores were later rectified, averaged, and used as the dependent variable in our multilevel analyses. Real-life social contact at the time of the e-diary prompt was assessed via an established binary scale that asks participants whether or not they are in the company of others.

The trained neural network can be maintained both on a local processor (such as a person's smartphone) and/or in virtual data storage. If the model is (also) maintained locally it will periodically be transferred to virtual storage to provide an updated digital mind twin 40 both locally and in virtual space.

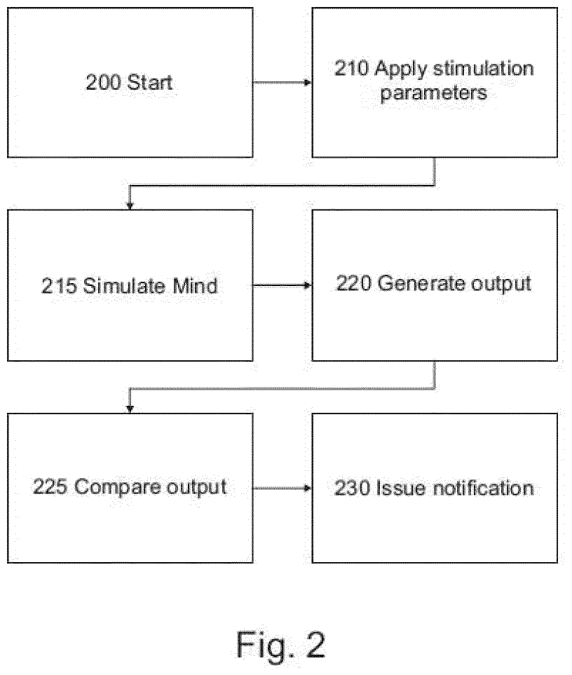

A flow diagram for a computer-implemented method for simulating a mental state of a mind of a human is shown in FIG. 2. The method starts in step 200 and comprises applying in step 210 one or more simulation parameters to the digital twin 40. The digital twin 40 is pre-programmed, as described above. The stimulation parameters could be one of more of readings from the plurality of physiological sensors 80 and/or one or more of readings of the environmental sensors 90.

The digital twin 40 simulates in step 215 the mind of the human, and the one or more simulation parameters 43 cause the digital twin 40 in step 220 to generate a digital twin output 45 that simulates behavior of the mind of the human in response to the one or more simulation parameters 45 and a notification sent in step 230.

Examples of the digital twin output 45 include an indication or prediction of early signs of mental health issues, such as predicting a lapsing of alcohol abuse, a promotion by mental health behaviors by graphically displaying to the user a connection between the mood and their level of physical activity or social interaction, furthering such behaviors, or the output of mental state parameters to inform the facial and bodily expression of the avatar representing the human user in a metaverse.

In one aspect, the mental data store 27 can include data about healthy humans and the method includes comparing in step 225 the digital twin output 45 with a response of a healthy human. This comparison can be used to improve the indications/predictions of mental health issues. Notifications 64 in step 230 are sent to the humans and/or their physicians or caregivers using a text message or a phone call to enable interventions. In another aspect, the digital twin output 45 can be passed on to an external application or platform. This could be a graphics engine 62 to generate the emotion-linked expressions on an avatar for the human, or an application that uses outputted mental state parameter to target a therapeutic intervention or propose a service or product.

In addition, the digital twin 40 can be used to simulate psychotherapeutic interventions and other forms of social interactions to aid in planning of treatment of the human, based on the predictive properties of the trained neural network representing mental state parameters.

### REFERENCE NUMERALS

10 Computer system
20 Memory
23 Plurality of instructions
25 Virtual data storage
27 Mental data storage
30 Processor
40 Digital twin
43 Stimulation parameters
45 Digital twin output
50 Models
60 Graphical user interface
62 Graphics engine
64 Notifications
70 Notification devices
80 Physiological Sensors
90 Environmental sensors

1. A computer-implemented system for simulating a state of a mind of a human, the system comprising:
a memory including a plurality of instructions, virtual data storage and mental data storage, the mental data storage storing human mental data relating to the mental state of the mind of the human;
at least one processor to execute ones of the plurality of instructions to generate at least:
a digital twin of the mind of the human, the digital twin including one or more models of the mind of the human based on mental data, the one or more models being stored in the virtual data storage and enabling analysis of the mind of the human by the at least one processor within the virtual data storage, and wherein the digital twin includes a link between the mental data storage and the virtual data storage.

2. The computer-implemented system of claim 1, further comprising a graphical user interface to provide visualization of and access to the human mental data based on an output from the digital twin.

3. The computer-implemented system of claim 1, further comprising one or more notification devices for triggering a notification from form the digital twin.

4. The computer-implemented system of claim 1, further comprising a prediction device for predicting the mental state of the human.

5. The computer-implemented system of claim 1, further comprising a graphics engine for generating an avatar, wherein the graphics engine is connected to the digital twin to enable generation of an avatar representative of the state of mind of the human.

6. A computer-implemented method for simulating a mental state of a mind of a human, the method comprising:
applying one or more simulation parameters to a digital twin, wherein the digital twin simulates the mind of the human, and wherein the one or more simulation parameters cause the digital twin to generate a digital twin output that simulates behavior of the mind of the human in response to the one or more simulation parameters.

7. The computer-implemented method of claim 6, wherein the digital twin output comprises a mental health prediction.

8. The computer-implemented method of claim 6, further comprising comparing the digital twin output with a response of a healthy human.

9. The computer-implemented method of claim 6, further comprising a generation of a notification about the output.

10. The computer-implemented method of claim 6, further comprising generation of an avatar based on data in the digital twin.

11. A computer program product stored in a memory and comprising a plurality of instructions to enable a processor to execute the computer-implemented method of claim 6.

12. A method for training a neural network to create a digital twin comprising:
inputting into a neural network one or more datasets characteristic of a mental state of a human; and
training the neural network from the one or more datasets.
